(19) United States
(12) Patent Application Publication
(10) Pub. No.: US 2017/0097330 A1
(43) Pub. Date: Apr. 6, 2017

(54) HYBRID ANALYZER FOR FLUID PROCESSING PROCESSES

(71) Applicant: Honeywell International Inc., Morristown, NJ (US)

(72) Inventor: JOHN LAWRENCE MANN, TORONTO (CA)

(21) Appl. No.: 14/875,377

(22) Filed: Oct. 5, 2015

(51) Int. Cl. G01N 33/28; G01N 21/359; G01N 21/65; G01N 21/33; G01N 21/3577

(52) U.S. Cl. CPC G01N 33/2829; G01N 21/33; G01N 21/3577; G01N 21/65; G01N 21/359

(57) ABSTRACT

A combined dataset is provided including (i) online process measurement data (OPMD), calculated process data derived from the OPMD or inline property data for liquid component(s) provided to the process from laboratory test results or an online analyzer (process measurement data), (ii) spectra data from online spectral analyzing a liquid product or offline spectra data from liquid product samples, and (iii) laboratory property data (LPD) of ≧1 product property from the product samples. When the spectra data is online data the dataset includes the OPMD and the online spectra data is coincidentally collected with product samples for the LPD. When the spectra data is offline spectra data the OPMD is coincidentally collected with the product samples for the LPD and for the offline spectra data. Using a Multivariate Data Analysis algorithm, a hybrid mathematical analyzer model is generated from the dataset which predicts the product property(ies) from (i) and (ii).

### FIELD

Disclosed embodiments relate to analyzer models for fluid processing processes

### BACKGROUND

Fluid processing, such as blending, distillation, reaction, evaporation, and filtration involves one or more fluids. The fluids can comprise transportation and heating fuels, components and additives used in manufacture of such fuels, chemicals including feedstocks and intermediates used in their manufacture, and water/wastewater streams.

Some process plants such as fuel refineries commonly employ spectrum analyzers (e.g. near-infrared analyzers (NIR), Raman analyzers) to monitor product properties during processing to better control operations. Such spectrum analyzers employ mathematical models (a set of correlations) to predict the values of properties from the spectra data as if they had been determined from rigorous (e.g. ASTM) test methods. Properties predicted are typically those named in product specifications for different fuel grades, e.g., gasolines, diesels, jet fuels, such as Octane (research octane number RON, motor octane MON), Reid Vapor Pressure, Density, Benzene %, Cloud Point, Freeze Point, Cetane Number, and Cetane Index. It is often found that the mathematical models do not perform adequately when operations change over time and many sites have difficulty updating them. Poor models can result in suspension of online optimization and increased product quality giveaway raising cost of production.

A typical practice for generating analyzer models is for a refinery or process site to provide many (e.g., 30 to 100) product samples obtained over a period of time to their analyzer vendor who applies a technique such as Partial Least Squares Regression (PLSR) to generate a mathematical analyzer model which correlates the spectra analysis of each sample to its measured properties. To improve model performance it is known to create multiple models for different grades of each product, different recipes or formulations of a product, or for different operating conditions in processing. This increases the cost of developing and maintaining the models.

The decision of how many analyzer models to develop for what grades/recipes is a mixture of art and science. Collecting samples is typically an arduous exercise. If samples of actual fuel blends are taken, a long time period is required as seasonal grades may not be blended for months. If samples are prepared by blending components in the lab, then normal component variability is not happening. Attempts to shrink the modelling effort by using fewer samples can result in analyzer models that perform poorly with only slight changes to recipes or components. The time and effort savings can be counterproductive since the exercise would have to be repeated more frequently to maintain blend optimization benefits.

Another method of determining product properties commonly used in process plants is to employ Inferential Analyzers. These are virtual analyzers that comprise mathematical models using measurements of selected operating conditions as inputs to calculate properties of a product as outputs. Development of Inferential Analyzer models also involves gathering sets of operating conditions and laboratory test results for the properties in question and employing a technique like partial least squares regression to develop the correlations. These models also suffer in accuracy when significant changes in operating conditions or feedstocks occur.

Inferential analyzer models are typically developed and maintained by process control engineers or consultants. An alternative to the inferential method that also uses process values as inputs to predict properties is to employ equations either developed from first principles or engineered from extensive testing where the structure of the equations are generally accepted to represent the true behavior of the system. Examples of such equations may be found in Chapter 3 of Characterization and Properties of Petroleum Fractions (M. R. Riazi, ASTM Stock Number: MNL50) for predicting properties of a blend based on the fractions of components in the blend and the properties of the individual components.

### SUMMARY

This Summary is provided to introduce a brief selection of disclosed concepts in a simplified form that are further described below in the Detailed Description including the drawings provided. This Summary is not intended to limit the claimed subject matter's scope.

Disclosed embodiments include method of generating hybrid analyzer mathematical models (hybrid analyzer mathematical models) for fluid processing processes by applying a Multivariate Data Analysis (MDA) algorithm to a disclosed combined dataset (dataset) that includes both process measurement data and spectra data. The dataset includes (i), (ii) and (iii). (i) comprises at least one of online process measurement data (OPMD) from instrument readings, calculated process data derived from the OPMD, and inline property data for at least one liquid component provided to the fluid process obtained from laboratory test results or from an online analyzer (process measurement data) (ii) comprises spectra data being online spectra data obtained from online spectral analyzing a liquid product of the fluid process or offline spectra data from samples of the liquid product (liquid product samples), and (iii) comprises laboratory property data (LPD) of at least one product property from the liquid product samples.

When the spectra data is online spectra data the dataset includes the OPMD and online spectra data coincidentally collected with the liquid product samples for the LPD at a plurality of different instants in time. When the spectra data is offline spectra data (e.g., laboratory spectra data) the OPMD is coincidentally collected with the liquid product samples for the LPD and for the offline spectra data at a plurality of different instants in time. Using a computing device having an associated memory implementing a Multivariate Data Analysis (MDA) algorithm, a hybrid mathematical analyzer model (hybrid analyzer model) is generated from the dataset which predicts the product property from (i) and (ii). Disclosed hybrid analyzer models thus effectively hybridize (combine) the data used by inferential analyzers and the data used by spectral analyzers.

A “Fluid Product” as used herein is defined as a product generated by a fluid processing process (blending, distillation, reaction, evaporation, filtration) involving one or more fluids, including but not limited to, transportation and heating fuels, components and additives used in manufacture of such fuels, chemicals including feedstocks and intermediates used in their manufacture, and water/wastewater streams. In some embodiments the fluid can be a fuel, such as gasoline or diesel fuel.

Disclosed hybrid analyzer models can be applied to a fuel mixing (or blending) or a fuel distillation process (fuel process). Applied to a fuel blending process, many product samples are collected from a plurality of different times and tested offline to verify the performance of the model used by the online analyzer (online analyzer model). Disclosed embodiments automatically capture online or generate offline spectra data together with offline laboratory data from product samples taken at the same time as the online data and retain that data as part of a blend or distillation monitoring application.

One can then access the data, provide this data to an MDA algorithm to generate a hybrid analyzer model that can be used to determine when the online analyzer model is out of tune, and generate new analyzer models that can be used to directly update the online analyzer model. Since spectra data (ii) and laboratory property data (iii) are collected together with process measurement data (i) about the blend or distilled product, for blending processes one can include additional data such as the blend recipe and component qualities in the hybrid analyzer model generation process. Inclusion of this additional data in generating disclosed hybrid analyzer models is expected to significantly improve the model's performance.

### BRIEF DESCRIPTION OF THE DRAWINGS

FIG. 1 is a flow chart that shows steps in an example method for example method for generating a disclosed hybrid analyzer model for predicting fluid product properties from a fluid processing process (fluid process), according to an example embodiment.

FIG. 2A shows in block form steps in an example method for generating a disclosed hybrid analyzer for analyzing products generated by a fluid processing process, according to an example embodiment.

FIG. 2B shows in block form data collection to provide a dataset for generation of a hybrid analyzer model.

FIG. 3 shows an example fuel blending system including a disclosed blend analysis tool showing the data flow involved with the blend analysis tool's generation of a new disclosed hybrid analyzer model for controlling the fuel mixing of a fuel blending process run by the fuel blending system, according to an example embodiment.

### DETAILED DESCRIPTION

Disclosed embodiments are described with reference to the attached figures, wherein like reference numerals are used throughout the figures to designate similar or equivalent elements. The figures are not drawn to scale and they are provided merely to illustrate certain disclosed aspects. Several disclosed aspects are described below with reference to example applications for illustration. It should be understood that numerous specific details, relationships, and methods are set forth to provide a full understanding of the disclosed embodiments.

One having ordinary skill in the relevant art, however, will readily recognize that the subject matter disclosed herein can be practiced without one or more of the specific details or with other methods. In other instances, well-known structures or operations are not shown in detail to avoid obscuring certain aspects. This Disclosure is not limited by the illustrated ordering of acts or events, as some acts may occur in different orders and/or concurrently with other acts or events. Furthermore, not all illustrated acts or events are required to implement a methodology in accordance with the embodiments disclosed herein.

Disclosed embodiments include methods of improving performance of spectral analyzers for online property measurement for fluid processing processes in process industries. A dataset is provided including (i), (ii) and (iii) as described above. Each record in the dataset applies to a single point in time where online data was captured at the same time as samples were collected for analysis. For each desired property, a MDA technique is used to generate a mathematical model to predict the value of the product property from the process measurement data (i) and spectra data (ii). This method amalgamates (combines) process measurement data (i) normally used to develop inferential analyzer models with spectra data (ii) normally used to develop spectral analyzer models into a single dataset. This dataset contains more information to account for property variations than either dataset alone and the resulting model generated is referred to as a hybrid analyzer model.

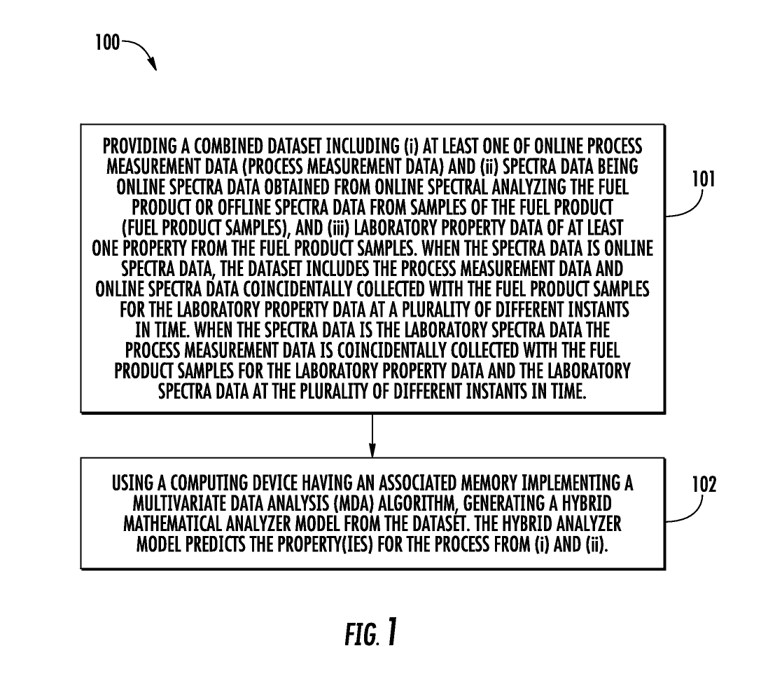

FIG. 1 is a flow chart that shows steps in an example method 100 for generating a disclosed hybrid analyzer model for predicting fluid product properties from a fluid processing process (fluid process), according to an example embodiment. Step 101 comprises providing a combined dataset (dataset) including (i) at least one of OPMD from instrument readings, calculated process data derived from the OPMD, and inline property data for at least one liquid component provided to the fluid process obtained from laboratory test results or from an online analyzer (process measurement data), (ii) spectra data being online spectra data obtained from online spectral analyzing a liquid product of the fluid process or offline spectra data from samples of the liquid product (liquid product samples), and (iii) laboratory property data (LPD) of at least one product property from the liquid product samples.

To use offline (e.g., bench) spectra data instead of online spectra data for method 100 the respective online and offline spectra analyzers should be properly maintained and calibrated, and the spectra data should be accessible. When the spectra data is online spectra data the dataset includes the OPMD and online spectra data coincidentally collected with the liquid product samples for the LPD at a plurality of different instants in time, and when the spectra data is offline spectra data (e.g., laboratory spectra data) the OPMD is coincidentally collected with the liquid product samples for the LPD and for the offline spectra data at the plurality of different instants in time.

Step 102 comprises using a computing device having an associated memory implementing a MDA algorithm, generating a hybrid mathematical analyzer model (hybrid analyzer model) from the dataset which predicts the product property(ies) from (i) and (ii). The MDA algorithm can act as a dimension-reduction methodology. For example, assuming an initial dataset of 20 instances of process measurement data (i) and 120 instances spectral data/absorbances (ii), the MDA algorithm can find correlations enabling a dataset reduction to 10 instances of process measurement data and 50 instances of spectral data/absorbances.

OPMD as used herein (including in i) refers to process measurement data obtained from sensors or gauges that measure inline process parameters involved in the process, for a blending fuel process being the measured percentage of each blend component and its components mixed in a blend which each individually have the same properties such as road octane number (RON), motor octane number (MON), Reid vapor pressure (RVP), as the blended product properties. The online or laboratory spectra data as used herein (including in ii) can be obtained from Near Infrared (NIR) spectroscopy, such as a set of transmittance vs. wavelength records. “Properties” as used herein in LPD (iii) in the case of a fuel process includes compositional properties such as % benzene, % Sulfur, Metals ppm, and intrinsic or intensive properties (e.g., Density, Conductivity, Cloud Point, Flash Point, Cloud Point, Octane Number, Cetane Number, Cetane Index) which describe petroleum fractions or products. Blending is a type of process with process measurement data, such as the percentage of each petroleum fraction used in the blend and with composition and property data of those petroleum fractions, as well as the final blended product.

The lab spectra data used herein (including in ii) can be obtained from a spectroscopy system (ultraviolet (UV), near-infrared (NIR)) or a Raman spectroscopy system. The laboratory property data can be obtained from physical property analyzers such as a gas chromatograph and/or a mass spectrometer. As noted above, the offline laboratory can also provide laboratory (bench) spectra data from the fuel product samples that can be used in the dataset used by the MDA algorithm.

The product property or properties can be defined in product specifications for the particular product, in the case of fuel being fuels including gasoline or diesel fuel. It is generally the same list for blended product as it is for component production in the case of distillation because it is usually the goal to control the production of components to have properties suitable to achieve the specifications in the blended product. Examples of properties for gasoline fuels include Research Octane Number, Motor Octane Number, Reid Vapor Pressure, Vapor Lock Index, Driveability Index, Distillation Temperatures (10%, 50%, 90% . . . ). Examples of properties for Diesel Fuels include Flash Point, Viscosity, Cetane Number, Cloud Point, Pour Point, and Density.

Partial Least Squares Regression (PLSR) and principle component regression (PCR) are both MDA methods to model a response (dependent) variable when there are a large number of predictor (independent) variables, and those predictors are highly correlated or even collinear. Both PLSR and PCR construct new predictor variables, known as components, as linear combinations of the original predictor variables. Factor analysis can also be used as the MDA method. Neural Nets is another technique which can be used.

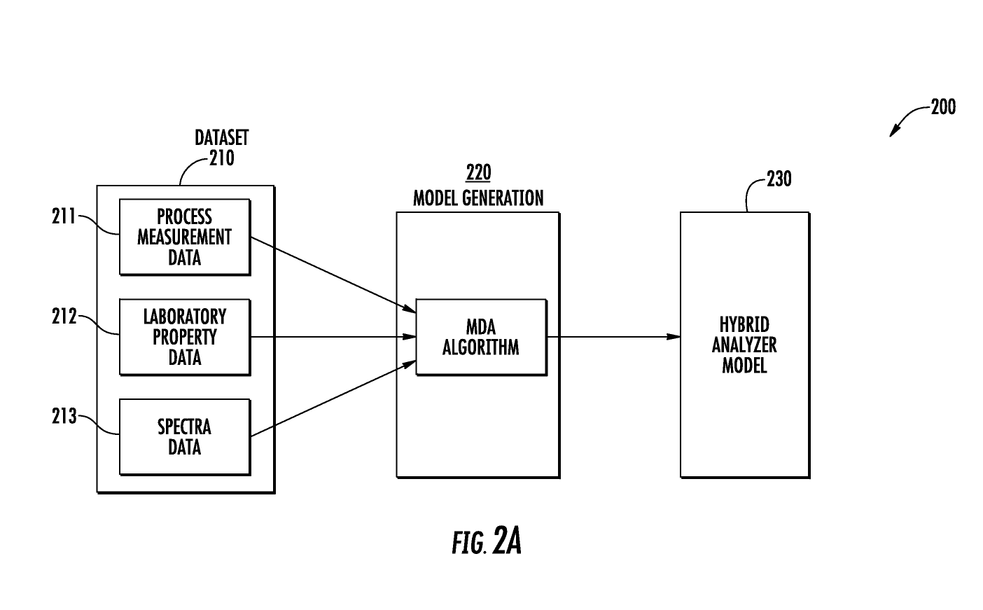

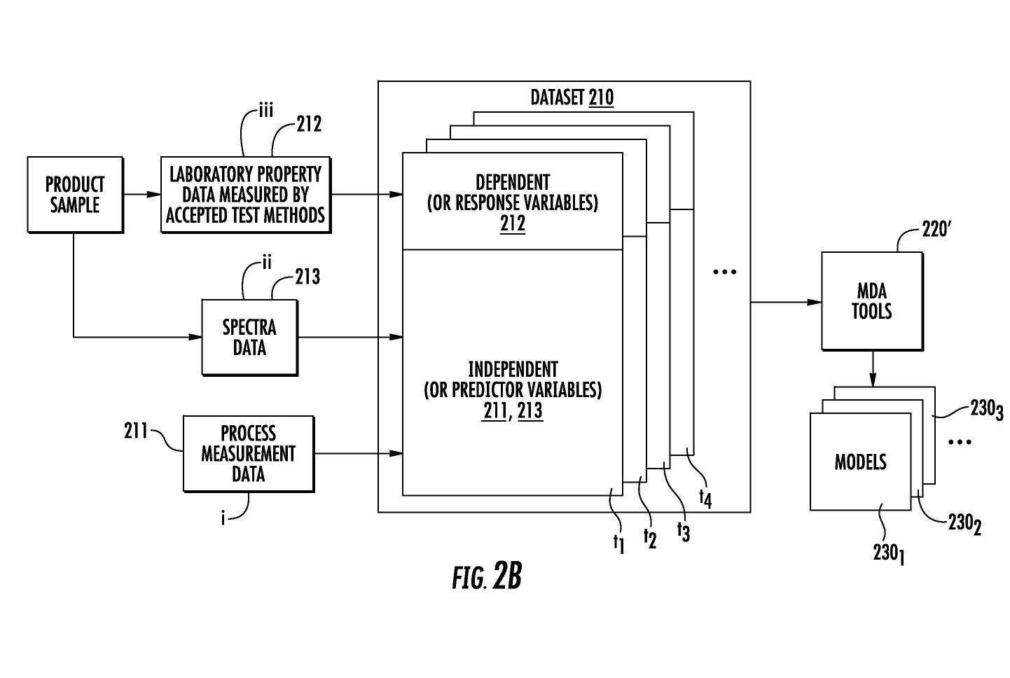

FIG. 2A shows in block form steps in an example method 200 for generating a disclosed hybrid analyzer for analyzing products generated by a fluid processing process, according to an example embodiment. Dataset block (dataset) 210 represents dataset collection. The dataset 210 includes process measurement data (i) 211, laboratory process data (LPD, iii) 212, and spectra data (ii) 213. As described above, the spectra data 213 can be online or offline spectra data, and the dataset includes the process measurement data and online spectra data coincidentally collected with the product samples for the laboratory property data at a plurality of different instants in time.

Block 220 represents model generation block involving application of the MDA algorithm to the dataset. Block 230 represents the output from the MDA algorithm block being a hybrid analyzer model.

FIG. 2B shows in block form an example data collection to provide a dataset including many different records (at different times) collected and assembled for generation of a hybrid analyzer model. From product samples spectra data (ii) 213, and LPD (iii) 212 are generated. From sensors or gauges in the fluid process, process measurement data 211 (i) is generated. The dataset 210 includes (i) 211 and 213 (ii) as independent (or predictor variables) and 212 (iii) as dependent (or response variables), each at a plurality of different instants in time shown as t1, t2, t3 and t4. An MDA tool 220′ acts on the dataset 210 to generate hybrid analyzer models 230₁, 230₂, 230₃ from the dataset 210 which predicts one or more product properties from (i) 211 and 213 (ii).

Product samples may be collected expressly for the purpose of generating a model with MDA tools, or they may be collected for the purpose of checking the performance of a model generated with MDA tools against results produced by accepted test methods. Such checking is commonly performed to apply corrections (e.g. bias updates) to a model generated with MDA tools to match analyzer property results to property results obtained by accepted test methods and use of such samples can significantly reduce the effort of data collection for model generation. It is noted that checking product samples yielding high corrections are likely to arise when the process is significantly different from the records used to build the MDA model currently in use and as such they are good candidates to include in generating a new model.

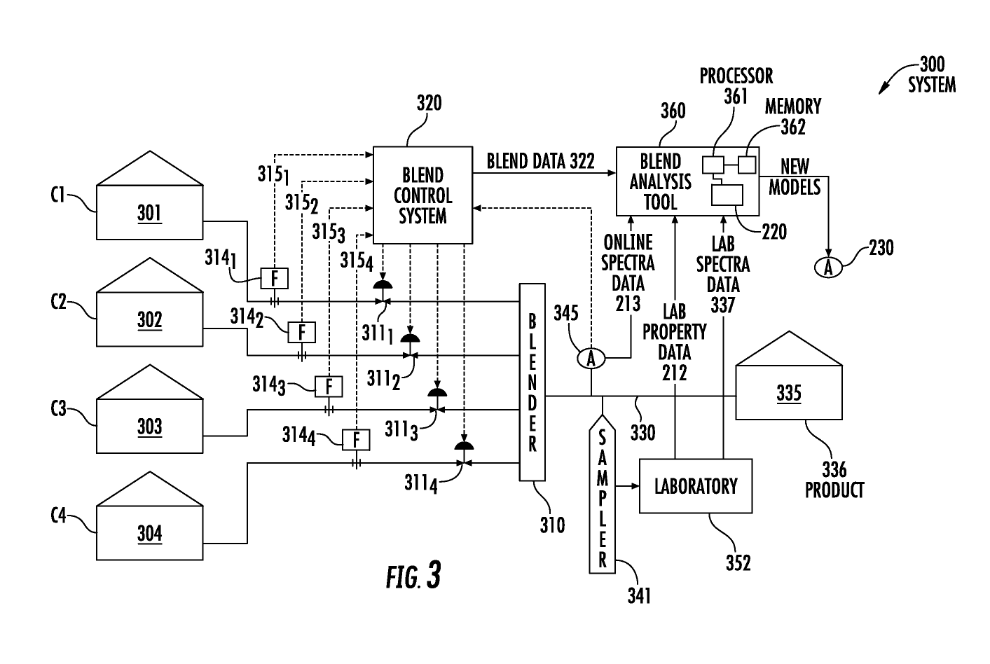

FIG. 3 shows an example fuel blending system 300 including a disclosed blend analysis tool 360 showing the data flow involved with the blend analysis tool's 360 generation of a new disclosed hybrid analyzer model shown as new model 230 from the dataset shown provided which predicts at least one fuel product property from fuel product generated by the fuel mixing process shown, according to an example embodiment. The blend analysis tool 360 shown represents a computing device that includes a processor 361 (e.g., digital signal processor (DSP), microcontroller unit (MCU) or field programmable gate array (FPGA)) having an associated memory 362 (on or off-chip) storing code for implementing the model generation block 220 shown which utilizes a MDA algorithm.

The four fuel components shown as C1, C2, C3 and C4 within respective tanks 301, 302, 303 and 304 are supplied to a blender 310 having respective feed lines that are each flow controlled by a flow valve 311₁ to 311₄. The flow valves 311₁-311₄ are each controlled independently by a process controller shown as a blend control system 320 that can comprise an Open Blend Property Controller (OPBC) provided by Honeywell International that may be referred to as a PROFIT® Blend Optimizer.

Blend control system 320 receives flow measurements 315₁ to 315₄ as its process measurement data from the respective flow sensors 314₁ to 314₄. Blend control system 320 can comprise a non-linear blend optimizer for online blend reformulation and optimization with dynamic recipe adjustment that allows efficient blending of fuels to required customer specifications while optimizing the blend. In this context, recognizing there may be a plurality of different recipes that may satisfy given product specifications, “optimizing” the blend refers to pushing the recipe based on a configured objective, such as the objective to minimize the cost of the components or to blend as closely to the product specifications as possible, or other objective(s). The blend control system 320 can use industry-standard open process control (OPC) server/client architecture to facilitate interfacing. The blender 310 outputs a blended fuel product (product) 336 that is coupled by an output line 330 to the input of a product tank 335.

The output line 330 is sampled to provide some fuel product 336 to a sampler 341 and to an online spectral analyzer (online analyzer) 345 which generates online spectra data 213 that is provided as an input to the blend analysis tool 360. The online spectra data 213 is also shown provided to the blend control system 320. The customer laboratory 352 comprises a bench spectral analyzer for providing offline (lab or bench) spectra data 337 to blend analysis tool 360 and physical property analyzers for providing laboratory property data 212 as well to the blend analysis tool 360. Sampler 341 provides fuel product samples from output line 330 to the laboratory 352.

The physical property analyzers of laboratory 352 analyzes samples of the fuel product 336 to generate laboratory property data 212 (e.g., road octane number (RON) or ROAD, motor octane number (MON)), Reid vapor pressure (RVP), density, Benzene, and Aromatics). The laboratory 352 also includes spectroscopy equipment for generating offline (lab) spectra data 337 similar to the online spectra data 213 provided by the online analyzer 345.

Blend analysis tool 360 can comprises a Honeywell International BLEND PERFORMANCE MONITOR. The blend analysis tool 360 comprises a decision support system that historizes all received data, highlights variances in the operation, and evaluates key performance indicators (KPIs) at the business level. The blend analysis tool 360 is also coupled to receive blend data 322 (e.g., flow measurements, a type of process measurement data, target files (blend files) containing the planned recipe for the blend, a start of blend file contains the recipe of what was actually set up on the distributed control system (DCS) to begin the blend, and an actual file contains the recipe that accrued during the blend from the blend control system 320.

Blend analysis tool 360 includes a model generation block 220 which applies a MDA algorithm of which PLSR is one example, to process the various data in the dataset received by the blend analysis tool 360. The dataset received by blend analysis tool 360 includes blend data 322 comprising cycle data that reflects flow measurements 315₁ to 315₄ as the process measurement data, laboratory property data including bench spectra analysis data 337, and online spectra 213 as spectra measurement data. Blend analysis tool 360 analyzes this dataset using a MDA algorithm and outputs the new hybrid analyzer model(s) 230.

The model used by online analyzer 345 and the hybrid analyzer model 230 are shown as being separate and thus different. However, the blend analysis tool's 360 hybrid analyzer model 230 output can connected to an input of the online analyzer 345 to allow the hybrid analyzer model 230 to be automatically provided to the online analyzer 345, although this transfer can be a manual transfer as well. Blend analysis tool 360 provides key performance indicators (KPI's) to monitor the model in current use by online analyzer 345, and if that model fails to perform up to expectation then a new model can be generated using the hybrid analyzer model 230 that can replace the current model for online analyzer 345. Monitoring of the new model used by the online analyzer 345 would generally be performed by blend analysis tool 360 as it is used.

The blend analysis tool 360 can employ statistical quality control (SQC) techniques to determine whether the model used by the online analyzer 345 needs tuning, and in that case to use the hybrid analyzer model 230 for the model generation for the analyzer model 345. The hybrid analyzer model 230 generated by blend analysis tool 360 can then be downloaded (automatically or manually) to the online analyzer 345 for use in generating a new analyzer model for online analyzer 345.

Fuel blending system 300 shown in FIG. 3 can be modified to enable realization of other systems, such as a distillation, reaction, evaporation, or filtration system. Processes such as distillation, reaction, evaporation, and filtration may be controlled by a distributed control system (DCS), and their implementation will generally have different measurements, controllers and processing equipment compared to those shown in FIG. 3 which are specific to the nature of the particular processes.

### EXAMPLES

Disclosed embodiments are further illustrated by the following specific Examples, which should not be construed as limiting the scope or content of this Disclosure in any way.

This Example involves hybrid analyzer model generation integrated with blend data from a fuel blending process. Assume for a particular blend application, one has process knowledge of what is occurring at the blender 310 that an analyzer model vendor would not have. This knowledge includes the component recipe and the component properties measured independently upstream of the blender (process measurement data 211). Laboratory property data 212 from samples collected from the particular blend that are tested in the refinery laboratory to check the online analyzer results is also provided.

At blend sample collection time(s) one can trigger the online analyzer 345 to write a file containing spectra (measurement) data 213 or the spectra data 213 may be generated using an analyzer in the laboratory 352. The dataset collected is a file including the spectra data 213 (spectra file), blend cycle data (process measurement data 211) and laboratory property data 212 from laboratory analysis of the fuel sample. One then generates a hybrid analyzer model using a blend analysis tool 360 that implements an MDA algorithm (e.g., based on PLS regression) from this dataset. The hybrid analyzer model being based on process measurement data 211, laboratory property data 212 and spectra data 213 is more robust and accurate as compared to a conventional inferential analyzer model (based only on process measurement data 211 and laboratory property data 212) or a conventional spectral analyzer model (based only on laboratory property data 212 and spectra data 213, that is typically online spectra data).

The component recipe data (percentage of components in the blend) as process measurement data 211 can be included in the MDA algorithm. This will produce one hybrid analyzer blend model that extends to different grades significantly better since the main contributor to grade differences is recognized to be the blend recipe. The component properties independently measured upstream of the blender 310 can also be included as a process measurement data 211 in the analysis performed by the MDA algorithm.

While various disclosed embodiments have been described above, it should be understood that they have been presented by way of example only, and not limitation. Numerous changes to the subject matter disclosed herein can be made in accordance with this Disclosure without departing from the spirit or scope of this Disclosure. In addition, while a particular feature may have been disclosed with respect to only one of several implementations, such feature may be combined with one or more other features of the other implementations as may be desired and advantageous for any given or particular application.

As will be appreciated by one skilled in the art, the subject matter disclosed herein may be embodied as a system, method or computer program product. Accordingly, this Disclosure can take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, this Disclosure may take the form of a computer program product embodied in any tangible medium of expression having computer usable program code embodied in the medium.

1. A method of generating models for a fluid processing process (fluid process), comprising:
providing a combined dataset (dataset) including (i) at least one of online process measurement data (OPMD) from instrument readings, calculated process data derived from said OPMD, and inline property data for at least one liquid component provided to said fluid process obtained from laboratory test results or from an online analyzer (process measurement data), (ii) spectra data being online spectra data obtained from online spectral analyzing a liquid product of said fluid process or offline spectra data from samples of said liquid product (liquid product samples), and (iii) laboratory property data (LPD) of at least one product property from said liquid product samples, when said spectra data is said online spectra data said dataset including said OPMD and said online spectra data coincidentally collected with said liquid product samples for said LPD at a plurality of different instants in time, and when said spectra data is said offline spectra data said OPMD is coincidentally collected with said liquid product samples for said LPD and for said offline spectra data at said plurality of different instants in time, and
using a computing device having an associated memory implementing a Multivariate Data Analysis (MDA) algorithm, generating a hybrid mathematical analyzer model (hybrid analyzer model) from said dataset which predicts said product property from said (i) and said (ii).

2. The method of claim 1, wherein said product property comprises a plurality of said product properties that are described on a product specification sheet for said liquid product.

3. The method of claim 1, wherein said liquid product comprises gasoline or diesel fuel.

4. The method of claim 3, wherein said fluid process comprises fuel blending.

5. The method of claim 1, further comprising using said hybrid analyzer model to update an online spectral analyzer.

6. The method of claim 1, wherein said (ii) is obtained using a spectroscopy system comprising ultraviolet (UV), near-infrared (NIR) or a Raman spectroscopy system.

7. The method of claim 1, wherein said MDA algorithm comprises partial least squares regression (PLSR), principle component regression (PCR) or Factor Analysis, said MDA algorithm reducing said process measurement data, said spectra data, and said laboratory property data to a smaller set of uncorrelated components as part a regression performed to provide said hybrid analyzer model.

8. A method of generating models for a fuel blending process, comprising:
providing a combined dataset (dataset) including (i) at least one of online process measurement data (OPMD) from instrument readings, calculated process data derived from said OPMD, and inline property data for at least one fuel component provided to said fuel blending process obtained from laboratory test results or from an online analyzer (process measurement data), (ii) spectra data being online spectra data obtained from online spectral analyzing a fuel product of said fuel blending process or offline spectra data from samples of said fuel product (fuel product samples), and (iii) laboratory property data (LPD) of at least one product property from said fuel product samples, when said spectra data is said online spectra data said dataset including said OPMD and said online spectra data coincidentally collected with said fuel product samples for said LPD at a plurality of different instants in time, and when said spectra data is said offline spectra data said OPMD is coincidentally collected with said fuel product samples for said LPD and for said offline spectra data at said plurality of different instants in time, and
using a computing device having an associated memory implementing a Multivariate Data Analysis (MDA) algorithm, generating a hybrid mathematical analyzer model (hybrid analyzer model) from said dataset which predicts said product property from said (i) and said (ii).

9. The method of claim 8, wherein said dataset further includes a blend recipe for said fuel blending process and qualities of said fuel component (component qualities), and wherein said blend recipe and said component qualities are utilized in said generating said hybrid analyzer model.

10. A fluid process analyzer tool, comprising:
a computing device having associated memory storing a Multivariate Data Analysis (MDA) algorithm for implementing said MDA algorithm;
wherein said MDA algorithm is for generating a hybrid mathematical analyzer model (hybrid analyzer model) from a combined dataset (dataset), said dataset including:
i) at least one of online process measurement data (OPMD) from instrument readings, calculated process data derived from said OPMD and inline property data for at least one liquid component provided to a fluid process obtained from laboratory test results or from an online analyzer (process measurement data), (ii) spectra data being online spectra data obtained from online spectral analyzing a liquid product of said fluid process or offline spectra data from samples of said liquid product (liquid product samples), and (iii) laboratory property data (LPD) of at least one product property from said liquid product samples, when said spectra data is said online spectra data said dataset including said OPMD and said online spectra data coincidentally collected with said liquid product samples for said LPD at a plurality of different instants in time, and when said spectra data is said offline spectra data said OPMD is coincidentally collected with said liquid product samples for said LPD and for said offline spectra data at said plurality of different instants in time,
wherein said hybrid analyzer model predicts said product property from said from said (i) and said (ii).

11. The analyzer tool of claim 10, wherein said product property comprises a plurality of said product properties that are described on a product specification sheet for said liquid product.

12. The analyzer tool of claim 10, wherein said liquid product comprises gasoline or diesel fuel.

13. The analyzer tool of claim 12, wherein said fluid process comprises fuel blending.

14. The analyzer tool of claim 10, wherein said MDA algorithm comprises partial least squares regression (PLSR), principle component regression (PCR) or Factor Analysis, said MDA algorithm reducing said process measurement data and said spectra data to a smaller set of uncorrelated components as part a regression performed to provide said hybrid analyzer model.

15. The analyzer tool of claim 10, further comprising an ultraviolet (UV), near-infrared (NIR) or a Raman spectroscopy system for obtaining said (ii).

16. The analyzer tool of claim 10, wherein said fluid process comprises a refinery process.
